(19) United States
(12) Patent Application Publication
(10) Pub. No.: US 2016/0169829 A1
(43) Pub. Date: Jun. 16, 2016

(54) METHOD AND APPARATUS FOR SENSING MOLECULAR GASES

(71) Applicant: CAMBRIDGE ENTERPRISE LIMITED, Cambridgeshire (GB)

(72) Inventors: MATTHEW PAUL HILLS, STAFFORD (GB); CARSTEN SCHWANDT, CAMBRIDGE (GB); VASANT KUMAR, CAMBRIDGE (GB)

(73) Assignee: ENVIROMENTAL MONITORING AND CONTROL LIMITED, STAFFORD (GB)

(21) Appl. No.: 14/907,770

(22) PCT Filed: Jul. 24, 2014
(86) PCT No.: PCT/GB2014/052267

§ 371 Date: Jan. 26, 2016

(30) Foreign Application Priority Data
Jul. 26, 2013 (GB) 1313396.2

(51) Int. Cl. G01N 27/407; G01N 33/00

(52) U.S. Cl. CPC G01N 27/4074; G01N 33/004; G01N 33/0042; G01N 33/0036

(57) ABSTRACT

A method and apparatus are provided for the quantitative sensing of molecular gases. The apparatus comprises a gas-sensitive measurement electrode (4,6), a series of solid ion-conductors including at least a salt ion-conductor (10) and a ceramic or glass ion-conductor (12), and a reference electrode (14, 16). The cell potential generated is a direct function of the pressure or concentration of the molecular gas to be sensed.

### FIELD OF INVENTION

The invention relates to a method and an apparatus for measuring the pressure or concentration of molecular gases, using a sensor that comprises a combination of solid ion-conductors in conjunction with a gas-sensitive measurement electrode, or auxiliary phase, and a reference electrode.

### BACKGROUND

The monitoring and control of the pressures or concentrations of molecular gases such as carbon oxides (CO₂, CO), sulphur oxides (SO₃, SO₂), nitrogen oxides (NO₂, NO), chlorine (Cl₂) and others is an important scientific and technological issue. There are a plethora of possible applications in the realms of environmental monitoring and process control. Examples include the measurement of CO₂ gas in the ambient atmosphere in the context of global warming, the measurement of CO₂ gas in buildings and cars in the context of on-demand ventilation control, and the measurement of various toxic gases in the context of monitoring combustion or metallurgical processes.

At present, the quantitative analysis of the gases mentioned is not straightforward. For quantitative CO₂ analysis, the predominantly applied method is infra-red spectroscopy. This requires bulky, sophisticated and expensive equipment and makes on-line analysis cumbersome. Even though very modern devices are now less bulky, they still cannot be miniaturised down to the size of typical electrochemical sensors. For quantitative SO₃ analysis, the only reliable method is wet chemical analysis. This involves complex and lengthy procedures and makes rapid on-line monitoring impossible. Spectroscopic methods can also be used but the additional complication is that the determination of small quantities of SO₃ in the presence of large quantities of SO₂ is critically affected by the similar absorption behaviours of both gases, rendering selective detection difficult. Problems of similar kinds are also encountered in the analysis of the other gases mentioned.

There is a pressing need to develop gas sensing systems for environmental monitoring and process control that should preferably be accurate and reliable, small and light, flexible to install and easy to use, as well as robust and affordable. These systems should also possess high sensitivity and selectivity as well as long-term stability and drift-free performance.

Attempts have been made to develop potentiometric sensors based on solid ion-conductors (solid electrolytes) to meet the requirements of a practical, commercial, gas-sensing system.

Sensors relying on solid ion-conductors with oxide ion or proton conductivity have become established technologies for the sensing of oxygen and hydrogen, respectively. Sensors using solid ion-conductors with metal ion conductivity are also known, with the majority of research efforts directed towards the sensing of CO₂ and SO₃, but no commercial breakthrough has as yet been achieved because all such sensors for molecular gases have failed to deliver adequate performance.

There are a large number of research articles on the subject of chemical sensors based on solid ion-conductors for use for sensing gases. Recent thorough and detailed review articles that define the state-of-the-art in the field are the following:
H.-H. Möbius, Galvanic solid electrolyte cells for the measurement of CO₂ concentrations, Journal of Solid State Electrochemistry 8 (2004) pp. 94-109.
J. W. Fergus, A review of electrolyte and electrode materials for high temperature electrochemical CO₂ and SO₂ gas sensors, Sensors and Actuators B 134 (2008) pp. 1034-1041.
P. Pasierb, M. R [symbol] kas, Solid-state potentiometric gas sensors—current status and future trends, Journal of Solid State Electrochemistry 13 (2009) pp. 3-25.

It is noted that the terms ‘pressure’ and ‘concentration’ are used interchangeably in relation to measurand gases throughout this document. Their conversion is possible via the ideal gas law p=(n R T)/V=c R T, where p is pressure, n is number of moles, R is the universal gas constant, T is absolute temperature, V is volume, and c is concentration.

Potentiometric Sensors

The potentiometric (or electrolytic, or galvanic) sensors described in the prior art for the sensing of gases such as CO₂, SO₃, NO₂ and Cl₂ using solid ion-conductors are based on the same fundamental sensor concept which comprises three distinct components: a measurement electrode and a reference electrode electrically connected through a solid ion-conductor (solid electrolyte).

More specifically, a typical solid-state potentiometric sensor used for the sensing of molecular gases comprises a gas-sensitive measurement electrode (or measuring electrode, or auxiliary phase, or auxiliary electrode) that equilibrates chemically with a target molecular-gas species in a measurand gas, a solid ion-conductor (solid electrolyte) that contains a mobile metal ion, and a reference electrode that provides a known, or predetermined, reference potential. The cell potential (cell voltage) is the difference between the electrode potential of the measurement electrode (or auxiliary phase) and the electrode potential of the reference electrode.

Solid Ion-Conductors

Solid ion-conductors (or solid electrolytes) are solid materials that possess a significant amount of mobile ions that render the materials ionically conducting. Solid ion-conductors may be divided into salt ion-conductors and ceramic ion-conductors. Also known are glass ion-conductors, but their properties are largely similar to those of the ceramic type and are not considered separately in the present context. Where appropriate in this document, for brevity, the term ceramic ion-conductor is used to include both ceramic and glass ion-conductors.

Salt solid ion-conductors have been known for over one hundred years and include, but are not limited to, materials such as sodium carbonate (Na₂CO₃), sodium nitrate (NaNO₃), sodium fluoride (NaF), sodium meta-silicate (Na₂SiO₃), sodium ortho-silicate (Na₄SiO₄), sodium phosphate (Na₃PO₄), sodium sulphate (Na₂SO₄), sodium chloride (NaCl), the analogous lithium (Li), potassium (K), rubidium (Rb), and caesium (Cs), compounds, as well as a number of analogous alkaline earth metal compounds, most importantly, calcium fluoride (CaF₂). Many more salt ion-conductors have been named in the literature and are known to those skilled in the art. The mobile ion in salt ion-conductors may be a metal ion (as in, for example, Na₂CO₃ Na₂SO₄, Na₂SiO₃) or a non-metal ion (as in, for example, CaF₂). This class of ion-conductor may be termed a salt ion-conductor or salt electrolyte even though a particular ion-conductor may be composite material, with the salt ion-conductor supported in or by a matrix such as a ceramic matrix.

Ceramic solid ion-conductors have been known for several decades and include, but are not limited to, materials such as fully or partially stabilised zirconia, partially substituted ceria, partially substituted perovskites, Li-β-alumina, Na-β-alumina, K-β-alumina, Mg-β-alumina, Ca-β-alumina, Sr-β-alumina, Ba-β-alumina, Cu-β-alumina, Ag-β-alumina, rare earth metal β-aluminas, NASICON, LISICON, CUSICON, and various oxide-based and fluoride-based glasses. Many more ceramic ion-conductors have been named in the literature and are known to those skilled in the art.

The ceramic solid ion-conductors with metal ion conductivity that are most widely applied are Na-β-alumina and NASICON. Na-β-alumina is a double oxide of Na₂O and Al₂O₃ with the general stoichiometry of Na₂O.xAl₂O₃ (x>1) and optional additions of stabilising oxides such as Li₂O or MgO. It is a two-dimensional ion-conductor, in which spinel blocks are bridged via oxygen atoms and the layers between the spinel blocks host the Na⁺ ions and allow for their fast transport. Specific compositions in this class of material are NaAl₅O₈ (Na-β″-alumina) and NaAl₁₁O₁₇ (Na-β-alumina). In the present context, the term ‘Na-β-alumina’ shall comprise any oxide from the Na—Al—O systems, including materials of the specific compositions above, materials of compositions deviating from the precise compositions above, mixtures of two or more materials of the above or deviating compositions, and materials with and without additional stabilising oxides. NASICON is a solid solution of Na₄Zr₂(SiO₄)₃ and NaZr₂(PO₄)₃ with the general stoichiometry of Na₁₊ₓZr₂SiₓP₃₋ₓO₁₂ (0<x<3). It is a three-dimensional ion-conductor, in which a large number of energetically similar filled and unfilled lattice sites exist that allow for fast Na⁺ ion transport. A specific composition with high Na⁺ ion conductivity is Na₃Zr₂Si₂PO₁₂.

This class of material may be termed ceramic ion-conductors or ceramic electrolytes.

In the following, a Na⁺ ion-conductor is used to exemplify the fundamental concept of gas sensing by means of solid metal-ion-conductors, but it is understood that analogous approaches are possible with other solid metal-ion-conductors.

Gas-Sensitive Measurement Electrodes

A gas-sensitive measurement electrode, or auxiliary electrode or auxiliary phase, comprises a material that is able to equilibrate chemically with a target species in an adjacent gas, and in particular a target species in a measurand gas comprising a mixture of at least the target gas species and another gas or gases, which may be termed a carrier gas or balance gas. The measurement electrode is in direct contact with a solid metal-ion-conductor and typically, but not necessarily, contains at least one compound of the metal whose ion is mobile in the solid metal-ion-conductor. Through this equilibration the chemical activity of this metal in the measurement electrode becomes uniquely defined. Four examples will be elaborated in greater detail as follows, for measuring CO₂, SO₃, NO₂ and Cl₂. Other systems are known to those skilled in the art.

The equilibration of Na₂CO₃ as the measurement electrode with CO₂ as the target species in the gas occurs via the following chemical equilibrium:

Na₂CO₃=2 Na+CO₂(g)+0.5 O₂(g) (1)

This equilibrium reaction fixes the chemical activity of Na in the Na₂CO₃. It is noted that not only CO₂ but also O₂ has an influence on the chemical activity of Na. Typically, in order to measure CO₂ concentration or pressure, the concentration or pressure of O₂ is measured separately by means of an O₂ sensor, so that the influence due to CO₂ can readily be singled out.

To measure SO₃ concentration or pressure, the equilibration of Na₂SO₄ as the measurement electrode with SO₃ as the target species in an adjacent or surrounding gas occurs via the following chemical equilibrium:

Na₂SO₄=2 Na+SO₃(g)+0.5 O₂(g) (2)

This equilibrium reaction fixes the chemical activity of Na in the Na₂SO₄.

The equilibration of NaNO₃ as the measurement electrode with NO₂ as the target species in an adjacent or surrounding gas occurs via the following chemical equilibrium:

NaNO₃═Na+NO₂(g)+0.5 O₂(g) (3)

This equilibrium reaction fixes the chemical activity of Na in the NaNO₃.

The equilibration of NaCl as the measurement electrode with Cl₂ as the target species in an adjacent or surrounding gas occurs via the following chemical equilibrium:

NaCl═Na+0.5 Cl₂(g) (4)

This equilibrium reaction fixes the chemical activity of Na in the NaCl.

In all of the above cases, the Na-containing compound in the measurement electrode (or auxiliary phase) equilibrates with the target species in the gas, which fixes the chemical activity of the Na in the measurement electrode. The Na in the Na⁺ ion-conductor in contact with the measurement electrode then equilibrates with the Na in the measurement electrode. This determines the electrode potential of the measurement electrode. Other measurement electrode materials may be used and analogous equilibria can be written with such compounds containing a different metal, and the ion corresponding to this different metal should then preferably be the mobile ion in the solid ion-conductor.

Reference Electrodes

A reference electrode is a material that provides a known, or predetermined, chemical activity of one of the elements involved in the sensor chemistry. The reference electrode is in direct contact (electrical contact) with a solid metal-ion-conductor and typically, but not necessarily, contains the metal, or at least one compound of the metal, whose ion is mobile in the solid metal-ion-conductor. Through the composition of the reference electrode the chemical activity of this metal in the reference electrode becomes uniquely defined.

Reference electrodes for metallic elements can be of various forms. The simplest one is the pure metal, protected from the gas (i.e. the measurand gas containing the target compound) by a hermetic seal. More complex ones are mixtures of compounds of which at least one contains the metal, and these are either protected from the gas by a seal or open to the gas. In the former case, species of the gas do not partake in the reactions in the reference electrode, while in the latter case they may do so. In all cases the compounds and phases in the reference electrode should preferably be chosen such that the chemical activity of the metal in the reference electrode becomes uniquely defined. Several examples will be elaborated in greater detail in the following, while many other systems are known to those skilled in the art.

For a Na reference electrode, the following systems have been described in the prior art: pure Na protected from the gas by means of a seal; a mixture of the two condensed phases Au and Au₂Na protected from the gas by means of a seal; a mixture of three condensed phases of the Na—Co—O or Na—Ni—O ternary systems protected from the gas by means of a seal; a mixture of two condensed phases and O₂ from the gas using systems such as Na₂ZrO₃, ZrO₂, O₂; Na₂MoO₄, MoO₃, O₂; Na₂WO₄, WO₃, O₂; Na₂SnO₃, SnO₂, O₂; Na₂Ti₆O₁₃, TiO₂, O₂; ZrSiO₄, ZrP₂O₇, O₂; Na₂Si₂O₅, SiO₂, O₂; Na₂Ti₆O₁₃, Na₂Ti₃O₇, O₂; Na₂Si₂O₅, SiO₂, O₂; Na₂Si₂O₅, Na₂SiO₃, O₂; Na₂Ge₄O₉, GeO₂, O₂.

When pure Na is used as the reference electrode, the chemical activity of Na in the reference electrode is unity. When multinary systems containing Na are used, the chemical activity of Na is fixed at values lower than unity. The Na in the Na⁺ ion-conductor in contact with the reference electrode equilibrates with the Na in the reference electrode. This determines the electrode potential of the reference electrode. It is understood that analogous equilibria can be written with compounds containing a different metal, and that the ion corresponding to this different metal should then preferably be the mobile ion in the solid ion-conductor.

Cell Potential

In order to measure the cell potential of a potentiometric sensor, the measurement electrode and the reference electrode are contacted with electronic conductors, typically of a noble metal, and the electronic conductors are connected to the two terminals of a voltmeter.

The cell potential arises from the difference in chemical activity, at the measurement electrode and at the reference electrode, of the element whose ion is mobile in the solid ion-conductor. The cell potential is given by the well-known Nernst equation:

$$U = - \frac{RT}{zF} ln \frac{{a}^{″}}{{a}^{′}} \qquad ( 5 )$$

where U is the cell potential, R is the universal gas constant of 8.3145 J mol⁻¹ K⁻¹, z is the number of elemental charges on the mobile ion (counted positive for cation-conductors and negative for anionconductors), F is the Faraday constant of 96487 C mol⁻¹, a″ is the chemical activity at the measurement electrode of the element whose ion is mobile in the solid ion-conductor, and a′ is the chemical activity at the reference electrode of this element.

For a known temperature and a known chemical activity a′ at the reference electrode, the measured cell potential is a direct function of the unknown chemical activity a″ at the measurement electrode. The chemical activity a″ in turn is a direct function of the pressure or concentration of the target species in the gas, consequent of the equilibration reactions (1) to (4). In this way the pressure or concentration of the target species is experimentally accessible.

In case the solid ion-conductor is a Na⁺ ion-conductor, the expression for the cell potential is:

$$U = - \frac{RT}{F} ln \frac{{a}_{Na}^{″}}{{a}_{Na}^{′}} \qquad ( 6 )$$

where a″_{Na} is the chemical activity of Na at the measurement electrode and a′_{Na} is the chemical activity of Na at the reference electrode.

Development of Sensors

Historically, the first solid state potentiometric sensors for the sensing of gases such as CO₂ and SO₃ involved salt solid metal-ion-conductors such as Na₂CO₃ and Na₂SO₄. As salt bodies are not mechanically robust and cannot be machined into complex shapes, the early sensor designs were assembled around salt discs and were operated as gas-concentration cells with different pressures or concentrations of the target species on both sides. However, this research line was soon abandoned as it became clear that no practically-useful devices could be constructed. Research then regained momentum with the advent of ceramic solid metal-ion-conductors. As these materials can be sintered and machined into diverse mechanically-stable shapes, sensor designs became more flexible and sensor assembly became simpler.

One possible potentiometric sensor for the combined sensing of CO₂ and O₂ gases comprises Na₂CO₃ as the gas sensitive measurement electrode, Na-β-alumina as the solid metal-ion-conductor, and Na of known chemical activity as the reference electrode. The galvanic cell symbol of this type of sensor is:

CO₂, O₂, Na₂CO₃|Na-β-alumina|Na (7)

where the reaction at the measurement electrode is:

Na₂CO₃=2 Na+CO₂(g)+0.5 O₂(g) (8)

The chemical activity of Na at the measurement electrode a″_{Na} is given through the chemical equilibration of the Na₂CO₃ of the measurement electrode with the CO₂ and O₂ in the gas. This activity can readily be shown to be:

$${a}_{Na}^{″} = \frac{{a}_{{Na}_{2} {CO}_{3}}^{1 / 2}}{{p}_{{CO}_{2}}^{1 / 2} {p}_{{O}_{2}}^{1 / 4}} exp \frac{Δ {G}_{{Na}_{2} {CO}_{3}}^{0} - Δ {G}_{{CO}_{2}}^{0}}{2 RT} \qquad ( 9 )$$

where p_{CO2} is the pressure of CO₂ in atm in the gas, p_{O2} is the pressure of O₂ in atm in the gas, a_{Na2CO3} is the chemical activity of Na₂CO₃ at the measurement electrode, and the ΔG⁰ values are the standard Gibbs free energies of formation of the compounds indicated.

The corresponding cell potential U is:

$$U = {U}^{0} - \frac{RT}{F} ln \frac{{a}_{{Na}_{2} {CO}_{3}}^{1 / 2}}{{p}_{{CO}_{2}}^{1 / 2} {p}_{{O}_{2}}^{1 / 4} {a}_{Na}^{′}} \qquad ( 10 )$$

where U⁰ is the known and constant standard electrode potential and a′_{Na} is the chemical activity of Na at the measurement electrode.

The cell potential can be simplified to:

$$U = {U}^{0} - \frac{RT}{F} ln \frac{1}{{p}_{{CO}_{2}}^{1 / 2} {p}_{{O}_{2}}^{1 / 4}} \qquad ( 11 )$$

in the case where pure Na₂CO₃ is used at the measurement electrode and pure Na is used at the reference electrode, so both these activities are unity.

The cell potential U depends on the pressures or concentrations of CO₂ and O₂ in the gas adjacent to the measurement electrode and the temperature. If p_{O2} is known or measured separately, and if the temperature is known, then p_{CO2} becomes a measurable quantity. It is understood that this sensing method rests on the assumption that the cell potential established is the one predicted by the Nernst equation.

One possible potentiometric sensor for the combined sensing of SO₃ and O₂ gases comprises Na₂SO₄ as the gas sensitive measurement electrode, Na-β-alumina as the solid metal-ion-conductor, and Na of known chemical activity as the reference electrode. The galvanic cell symbol of this type of sensor is:

SO₃, O₂, Na₂SO₄|Na-β-alumina|Na (12)

where the reaction at the measurement electrode is:

Na₂SO₄=2 Na+SO₃(g)+0.5 O₂(g) (13)

The chemical activity of Na at the measurement electrode a″_{Na} is given through the chemical equilibration of the Na₂SO₄ of the measurement electrode with the SO₃ and O₂ in the gas. This activity can readily be shown to be:

$${a}_{Na}^{″} = \frac{{a}_{{Na}_{2} {SO}_{4}}^{1 / 2}}{{p}_{{SO}_{3}}^{1 / 2} {p}_{{O}_{2}}^{1 / 4}} exp \frac{Δ {G}_{{Na}_{2} {SO}_{4}}^{0} - Δ {G}_{{SO}_{3}}^{0}}{2 RT} \qquad ( 14 )$$

where p_{SO3} is the pressure of SO₃ in atm in the gas, p_{O2} is the pressure of O₂ in atm in the gas, a_{Na2SO4} is the chemical activity of Na₂SO₄ at the measurement electrode, and the ΔG⁰ values are the standard Gibbs free energies of formation of the compounds indicated.

The corresponding cell potential U is:

$$U = {U}^{0} - \frac{RT}{F} ln \frac{{a}_{{Na}_{2} {SO}_{4}}^{1 / 2}}{{p}_{{SO}_{3}}^{1 / 2} {p}_{{O}_{2}}^{1 / 4} {a}_{Na}^{′}} \qquad ( 15 )$$

where U⁰ is the known and constant standard electrode potential and a′_{Na} is the chemical activity of Na at the measurement electrode.

The cell potential can be simplified to:

$$U = {U}^{0} - \frac{RT}{F} ln \frac{1}{{p}_{{SO}_{3}}^{1 / 2} {p}_{{O}_{2}}^{1 / 4}} \qquad ( 16 )$$

in case pure Na₂SO₄ is used at the measurement electrode and pure Na is used at the reference electrode, so both these activities are unity.

The cell potential U depends on the pressures or concentrations of SO₃ and O₂ in the gas adjacent to the measurement electrode and the temperature. If p_{O2} is known or measured separately, and if the temperature is known, then p_{SO3} becomes a measurable quantity. It is understood that this sensing method rests on the assumption that the cell potential established is the one predicted by the Nernst equation.

One possible potentiometric sensor for the combined sensing of NO₂ and O₂ gases comprises NaNO₃ as the gas sensitive measurement electrode, Na-β-alumina as the solid metal-ion-conductor, and Na of known chemical activity as the reference electrode. The galvanic cell symbol of this type of sensor is:

NO₂, O₂, NaNO₃|Na-β-alumina|Na (17)

where the reaction at the measurement electrode is:

NaNO₃═Na+NO₂(g)+0.5 O₂(g) (18)

The chemical activity of Na at the measurement electrode a″_{Na} is given through the chemical equilibration of the NaNO₃ of the measurement electrode with the NO₂ and O₂ in the gas. This activity can readily be shown to be:

$${a}_{Na}^{″} = \frac{{a}_{{NaNO}_{3}}}{{p}_{{NO}_{2}} {p}_{{O}_{2}}^{1 / 2}} exp \frac{Δ {G}_{{NaNO}_{3}}^{0} - Δ {G}_{{NO}_{2}}^{0}}{RT} \qquad ( 19 )$$

where p_{NO2} is the pressure of NO₂ in atm in the gas, p_{O2} is the pressure of O₂ in atm in the gas, a_{NaNO3} is the chemical activity of NaNO₃ at the measurement electrode, and the ΔG⁰ values are the standard Gibbs free energies of formation of the compounds indicated.

The corresponding cell potential U is:

$$U = {U}^{0} - \frac{RT}{F} ln \frac{{a}_{{NaNO}_{3}}}{{p}_{{NO}_{2}} {p}_{{O}_{2}}^{1 / 2} {a}_{Na}^{′}} \qquad ( 20 )$$

where U⁰ is the known and constant standard electrode potential and a′_{Na} is the chemical activity of Na at the measurement electrode.

The cell potential can be simplified to:

$$U = {U}^{0} - \frac{RT}{F} ln \frac{1}{{p}_{{NO}_{2}} {p}_{{O}_{2}}^{1 / 2}} \qquad ( 21 )$$

in case pure NaNO₃ is used at the measurement electrode and pure Na is used at the reference electrode, so both these activities are unity.

The cell potential U depends on the pressures or concentrations of NO₂ and O₂ in the measurement electrode adjacent to the gas and the temperature. If p_{O2} is known or measured separately, and if the temperature is known, then p_{NO2} becomes a measurable quantity. It is understood that this sensing method rests on the assumption that the cell potential established is the one predicted by the Nernst equation.

One possible potentiometric sensor for the sensing of Cl₂ gas comprises NaCl as the gas sensitive measurement electrode, Na-β-alumina as the solid metal-ion-conductor, and Na of known chemical activity as the reference electrode. The galvanic cell symbol of this type of sensor is:

Cl₂, NaCl|Na-β-alumina|Na (22)

where the reaction at the measurement electrode is:

NaCl═Na+0.5 Cl₂(g) (23)

The chemical activity of Na at the measurement electrode a″_{Na} is given through the chemical equilibration of the NaCl of the measurement electrode with the Cl₂ in the gas. This activity can readily be shown to be:

$${a}_{Na}^{″} = \frac{{a}_{NaCl}}{{p}_{{Cl}_{2}}^{1 / 2}} exp \frac{Δ {G}_{NaCl}^{0}}{RT} \qquad ( 24 )$$

where p_{Cl2} is the pressure of Cl₂ in atm in the gas, a_{NaCl} is the chemical activity of NaCl at the measurement electrode, and the ΔG⁰ value is the standard Gibbs free energy of formation of the compound indicated.

The corresponding cell potential U is:

$$U = {U}^{0} - \frac{RT}{F} ln \frac{{a}_{NaCl}}{{p}_{{Cl}_{2}}^{1 / 2} {a}_{Na}^{′}} \qquad ( 25 )$$

where U⁰ is the known and constant standard electrode potential and a′_{Na} is the chemical activity of Na at the measurement electrode.

The cell potential can be simplified to:

$$U = {U}^{0} - \frac{RT}{F} ln \frac{1}{{p}_{{Cl}_{2}}^{1 / 2}} \qquad ( 26 )$$

in case pure NaCl is used at the measurement electrode and pure Na is used at the reference electrode, so both these activities are unity.

The cell potential U depends on the pressure or concentration of Cl₂ in the gas adjacent to the measurement electrode and the temperature. If the temperature is known, then p_{Cl2} becomes a measurable quantity. It is understood that this sensing method rests on the assumption that the cell potential established is the one predicted by the Nernst equation.

The standard Gibbs free energy values ΔG⁰ required in the formulae above are available from tables and databases known to those skilled in the art, for example, JANAF Tables, Barin Knacke, or HSC Chemistry for Windows.

Problems

It should be noted that, despite the straightforward scientific concepts described above, the substantial volume of literature on the subject, and the huge demand for monitoring and control systems for molecular gases, no sensor of the above type has as yet proven to be viable in a practical application. The major problems observed by developers and users of such sensors are the following: the cell potential does not reach the thermodynamically-expected value according to the Nernst equation, nominally-identical sensors yield different cell potentials under the same experimental conditions, and the cell potential drifts with time.

### SUMMARY OF INVENTION

The present invention provides a method and apparatus for measuring, or for quantitatively sensing, the pressure or concentration of a molecular gas, such as CO₂, SO₃, NO₂ or Cl₂, as defined in the appended independent claims to which reference should now be made. Preferred or advantageous features of the invention are set out in dependent subclaims.

A first preferred embodiment of the invention may thus provide an apparatus comprising: a ceramic or glass, solid ion-conductor (or solid electrolyte) that contains a first metal ion as its mobile species; a salt ion-conductor (or solid electrolyte) in electrical contact with the ceramic, or glass, ion-conductor and that contains the first or a second, different, ion as its mobile species; a gas-sensitive measurement electrode in electrical contact with one of the ion conductors, the measurement electrode including an auxiliary phase comprising one or more chemical compounds that is or are able to equilibrate chemically with a target molecular species in a gas, and that contains at least one compound of the metal whose ion is mobile in the ion-conductor; and a reference electrode that provides a predetermined, or reference, chemical activity of the metal whose ion is mobile in the other ion-conductor and that is in electrical contact with the other ion-conductor. In this structure, the ceramic, or glass, ion-conductor and the salt ion-conductor form an electrolyte of the sensor between the electrodes.

A further preferred embodiment of the invention may thus provide an apparatus comprising: a first ion-conductor which is a ceramic or glass ion-conductor; a second ion-conductor which is a salt ion-conductor; a third ion-conductor which is a ceramic or glass ion-conductor; a gas-sensitive measurement electrode; and a reference electrode. The first, second and third ion-conductors contain first, second and third metal ions as their mobile species, and any of the first, second and third ion species may be the same as or different from each other. The second ion-conductor separates, and is in electrical contact with, the first and the third ion-conductors. The measurement electrode contains an auxiliary phase that comprises one or more chemical compounds that is or are able to equilibrate chemically with a molecular species in a gas (to which the measurement electrode is exposed during use of the sensor) and that contains at least one compound of the metal whose ion is mobile in the first ion-conductor. The measurement electrode is in electrical contact with the first ion-conductor. The reference electrode provides a predetermined, or reference, chemical activity of the metal whose ion is mobile in the third ion-conductor and is in electrical contact with the third ion-conductor. The second ion-conductor separates, and is in electrical contact with, the first and the third ion-conductors. In this structure, the ceramic, or glass, ion-conductors and the salt ion-conductor form an electrolyte of the sensor between the electrodes.

An apparatus of this type may advantageously be able to establish a thermodynamically-expected cell potential and/or not be subject to potential drift.

In other words, embodiments of the invention may advantageously provide a method and apparatus for the quantitative sensing of molecular gases. The apparatus may comprise a gas-sensitive measurement electrode, an electrically-connected series of solid ion-conductors including at least a salt ion-conductor and a ceramic or glass ion-conductor, and a reference electrode. The cell potential generated may then be a direct function of the pressure or concentration of the molecular gas to be sensed.

The inventors' current understanding is that the reason for the unsatisfactory performance of the sensors described in the prior art, which has not been appreciated in the prior art, lies in the occurrence of partial electronic conduction through the ceramic solid ion-conductors. This phenomenon causes cell potentials lower than theoretically-expected values. Electronic conduction across an ion-conductor between two electrodes may occur when the chemical activity of the element whose ion is mobile in the ion-conductor is outside the electrolytic domain of the ion-conductor at either or both electrodes, so that the material may no longer be considered a pure ion-conductor but must instead be considered a mixed ionic and electronic conductor. The precise ratio of ionic conductivity to electronic conductivity may depend on various factors related to the properties of the solid conductor, the composition of the surrounding gas and the cell (or sensor) geometry, and may vary between individual experimental arrangements. This understanding accounts readily for the generally-observed lack of consistency and reproducibility described in the prior art.

The inventors have surprisingly found that the problem of experimentally-measured cell potentials being lower than the theoretically-expected ones may be overcome by a novel and unique modification of the galvanic cells employed. This modification consists in arranging in series a ceramic solid ion-conductor and a salt ion-conductor, or a first ceramic solid metal-ion-conductor, a salt solid ion-conductor, and a second ceramic solid metal-ion-conductor, and placing this arrangement in between a gas-sensitive measurement electrode and a reference electrode. The measurement electrode and/or the reference electrode may be conventional electrodes, from conventional galvanic cell systems.

Such an arrangement has been found in the inventors' experiments to establish the theoretically-expected cell potentials and to show no cell potential drift.

In a preferred embodiment of the invention, two ceramic solid metal-ion-conductors are physically separated by a salt solid ion-conductor. This arrangement produces good performance and is advantageously straightforward to manufacture and to package.

Without the invention being limited to the following theory, the inventors believe that the salt solid ion-conductor changes the transference properties of the ionic and electronic charge carriers across the galvanic cell. This occurs in such a way that the ionic conductivity in each cell component remains sufficient to enable the build-up of a measurable cell potential, but the electronic conductivity across the entire cell is diminished to such an extent that deviations from the theoretical cell potential become small or negligible.

To achieve more rapid measurements, the physical dimensions of a sensor embodying the invention may be made a small as possible, and in particular the thickness of the salt ion-conductor, as well as the thicknesses of the ceramic ion-conductors, may be made as small as possible. In practice, the inventors have found that the thickness of the salt ion-conductor is of particular importance. In addition, to increase the speed of measurement, the temperature of the sensor may be raised. Advantageously, a minimum operating temperature of the sensor may be 300° C., but more rapid measurement may be achieved at temperatures above 450° C. If measurements are being made in ambient temperatures within these ranges, then rapid measurements may be achieved. If measurements are to be made in lower ambient temperatures, then a sensor may be heated, for example electrically heated, while measurements are made. Sensors embodying the invention may advantageously be small in size, so that little energy is required to heat a sensor to a desired measuring temperature range.

### SPECIFIC EMBODIMENTS

Specific embodiments of the invention will now be described by way of example, with reference to the accompanying drawings, in which:

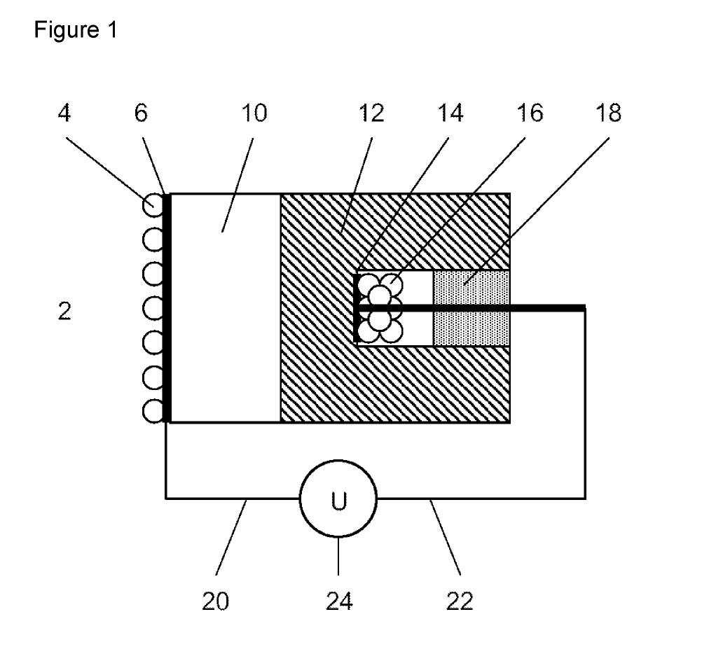

FIG. 1 is a schematic cross-section of a first embodiment of the invention in the form of a galvanic cell for the quantitative sensing of molecular gases;

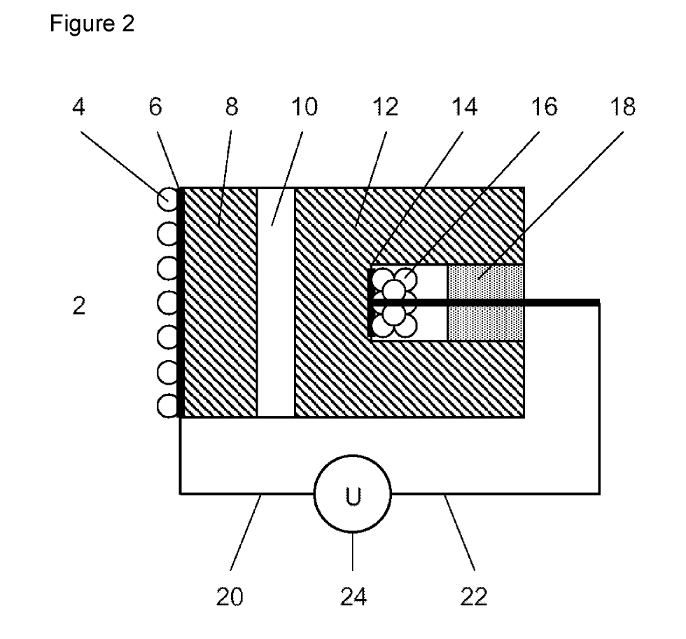

FIG. 2 is a schematic cross-section of a second embodiment of the invention in the form of a galvanic cell for the sensing of molecular gases;

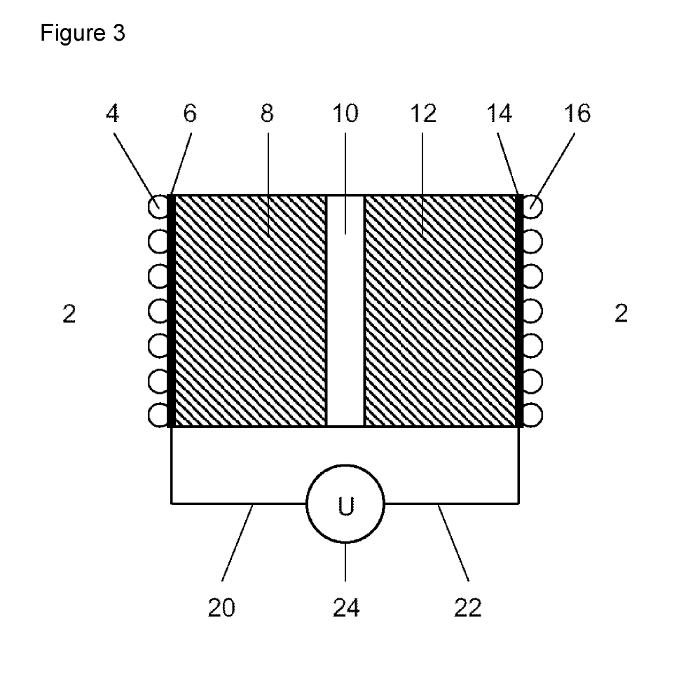

FIG. 3 is a schematic cross-section of a third embodiment of the invention in the form of a galvanic cell for the sensing of molecular gases;

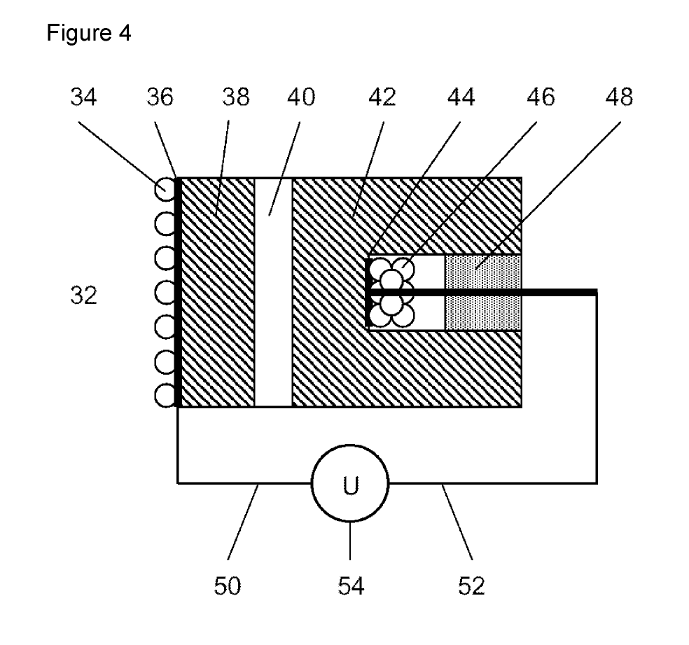

FIG. 4 is a schematic cross-section of a fourth embodiment of the invention in the form of a galvanic cell for the sensing of CO₂ and O₂ gases;

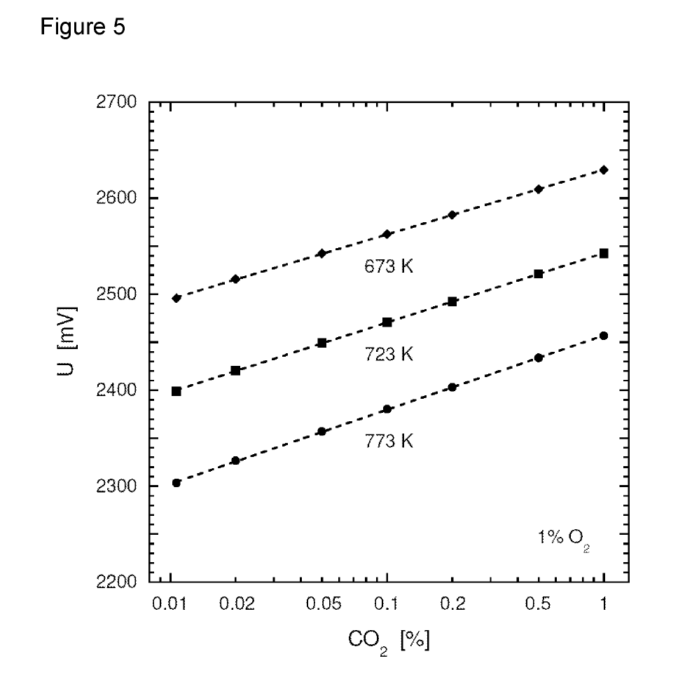

FIG. 5 is a graph showing the cell potential of the cell in FIG. 4 as a function of the CO₂ concentration of a measured gas;

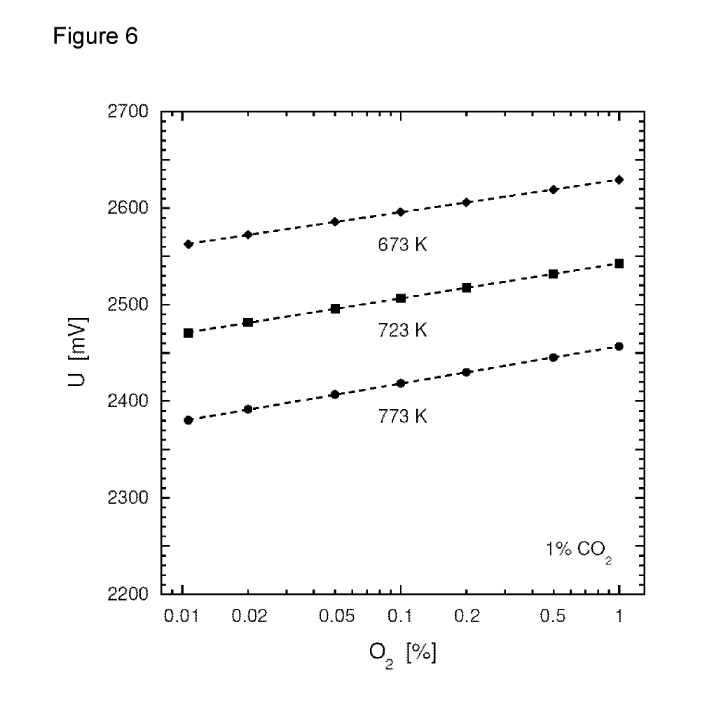

FIG. 6 is a graph showing the cell potential of the cell in FIG. 4 as a function of the O₂ concentration of the measured gas;

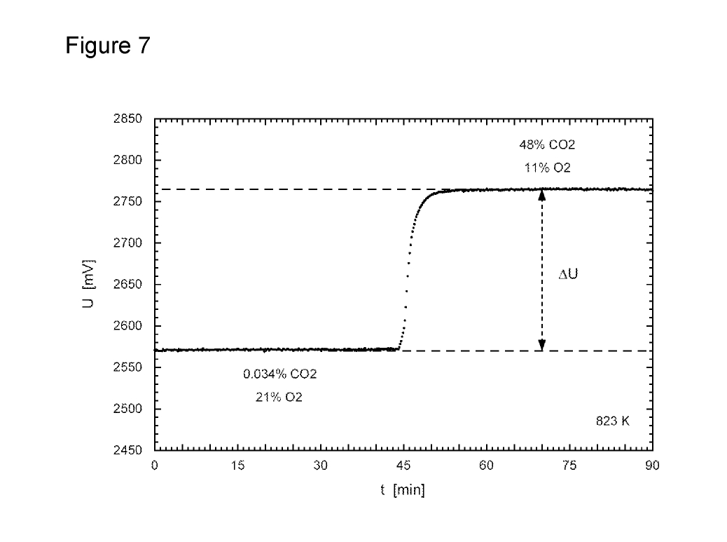

FIG. 7 is a graph showing the time-dependent cell potential of the cell in FIG. 4 when responding to a variation of the CO₂ and O₂ concentrations of the measured gas;

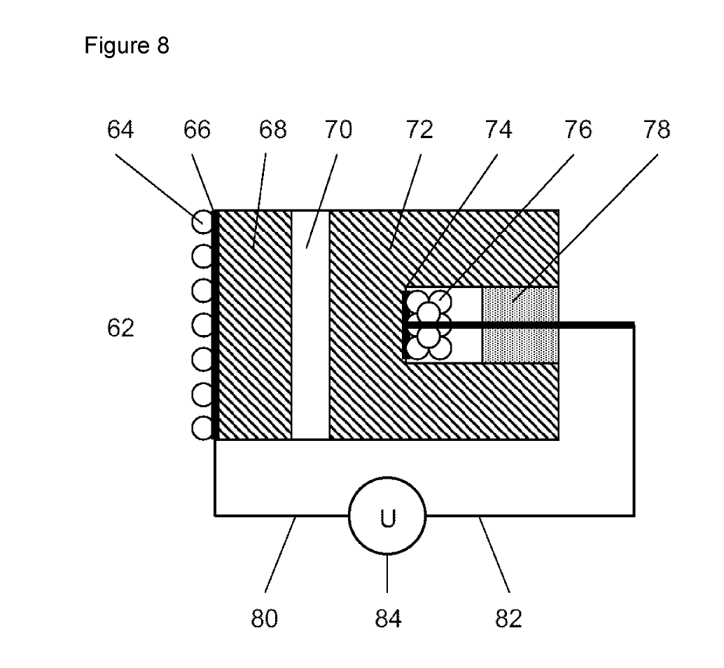

FIG. 8 is a schematic cross-section of a fifth embodiment of the invention in the form of a galvanic cell for the sensing of SO₃ and O₂ gases; and

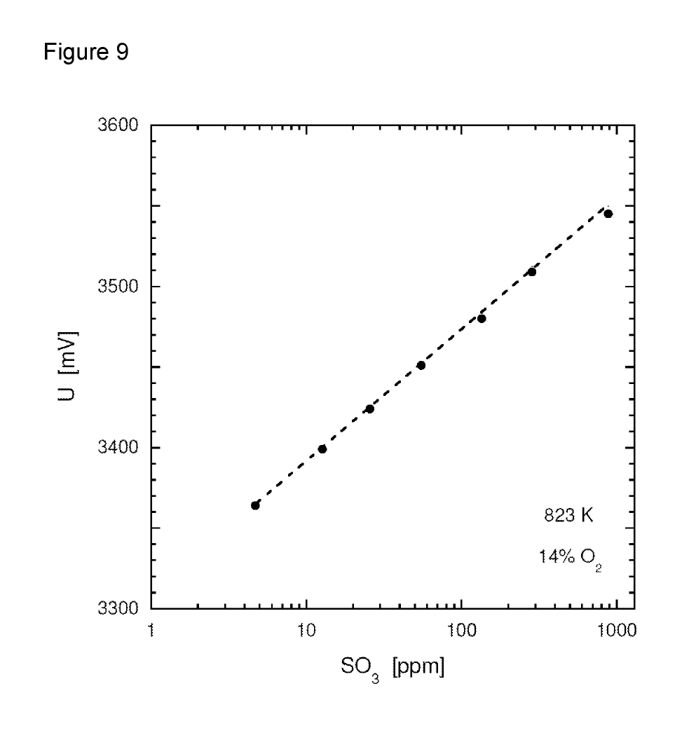

FIG. 9 is a graph showing the cell potential of the cell in FIG. 8 as a function of the SO₃ concentration of the measured gas.

FIG. 1 is a schematic cross-section of a first embodiment of the invention in the form of a galvanic cell for the sensing of molecular gases. This shows the gas 2 and, electrically connected in series, a gas-sensitive measuring electrode 4, a first electronically-conducting contact 6, a salt solid ion-conductor 10, a ceramic solid metal-ion-conductor 12, a second electronically conducting contact 14, and a reference electrode 16 that is not open to the gas. The reference electrode is contained in a blind-ended recess in the ceramic ion-conductor and is closed from the gas by a seal 18. A metal lead to the first electronically-conducting contact 20 and a metal lead to the second electronically-conducting contact 22 are connected to an electrometer or voltmeter 24 for making measurements.

FIG. 2 is a schematic cross-section of a first embodiment of the invention in the form of a galvanic cell for the sensing of molecular gases. The same reference numerals are used in FIG. 2 as in FIG. 1, where appropriate. FIG. 1 shows the gas 2 and, electrically connected in series, a gas-sensitive measurement electrode 4, a first electronically-conducting contact 6, a first ceramic solid metal-ion-conductor 8, a salt solid ion-conductor 10, a second ceramic solid metal-ion-conductor 12, a second electronically-conducting contact 14, and a reference electrode 16 that is not open to the gas. The reference electrode is contained in a blind-ended recess in the second ion-conductor and is closed from the gas by a seal 18. A metal lead to the first electronically-conducting contact 20 and a metal lead to the second electronically-conducting contact 22 are connected to an electrometer or voltmeter 24 for making measurements.

As described below, in embodiments of the invention the salt ion-conductor 10 may be formed in various ways, including as a separate component or as a surface layer on one or both of the adjacent ceramic ion-conductors.

FIG. 3 is a schematic cross-section of a second embodiment of the invention in the form of a novel galvanic cell for the sensing of molecular gases. This includes the gas 2 and, electrically connected in series, a gas sensitive measurement electrode 4, a first electronically-conducting contact 6, a first ceramic solid metal-ion-conductor 8, a salt solid ion-conductor 10, a second ceramic solid metal-ion-conductor 12, a second electronically-conducting contact 14, and a reference electrode 16 that is open to the gas. A metal lead to the first electronically-conducting contact 20 and a metal lead to the second electronically-conducting contact 22 are connected to an electrometer 24.

FIG. 4 is a schematic cross-section of a third embodiment of the invention in the form of a galvanic cell for the sensing of CO₂ and O₂ gases. The structure of the sensor is the same as in FIG. 1, but in this case the gas 32 contains CO₂ and O₂, the gas sensitive measurement electrode is Na₂CO₃ 34 which is exposed to the gas, and the electronically-conducting contact at the measurement electrode is platinum or gold 36. The chemical activity of Na in the measurement electrode arises from its equilibration with the CO₂ and O₂. The combination of solid ion-conductors comprises a first Na-β-alumina body 38, a Na₂SO₄ body 40, and a second Na-β-alumina body 42. The electronically-conducting contact at the reference electrode is platinum, gold, tungsten, steel, Inconel, a combination thereof, or some other suitable refractory electronic conductor 44, the reference electrode is pure Na metal 46 which is separated from the environment by means of a glass seal 48. The chemical activity of Na in the reference electrode is unity. The metal leads, or lead wires, coupling the electronically-conducting contacts of the two electrodes to the electrometer 54 are of platinum, gold, tungsten, steel, Inconel, a combination thereof, or some other suitable refractory electronic conductor 50, 52. The Na-β-alumina body 42 that contains the reference electrode is gastight. In other embodiments, the Na₂SO₄ body 40 may be replaced with a body of Na₂CO₃, Na₂SiO₃, NaF, NaCl, CaF₂, or some other suitable aforementioned material.

FIG. 5 is a graph showing the cell potential of the cell in FIG. 4 as a function of the CO₂ concentration of a measured gas. In experiments, the CO₂ concentration was varied between 0.01 and 1% by volume, the O₂ concentration was fixed at 1% by volume, the balance gas was argon, and the temperature was varied between 400 and 500° C. (673 and 773 K). The solid symbols in the graph represent measured values and the dashed lines represent thermodynamically-expected values. The cell provided potentials that coincided to within +/−1 mV with the theoretical values, irrespective of whether a body of Na₂SO₄, Na₂CO₃, Na₂SiO₃, NaF, NaCl or CaF₂ was used in between the two Na-β-alumina bodies.

FIG. 6 is a graph showing the cell potential of the cell in FIG. 4 as a function of the O₂ concentration of the measured gas. In experiments, the O₂ concentration was varied between 0.01 and 1% by volume, the CO₂ concentration was fixed at 1% by volume, the balance gas was argon, and the temperature was varied between 400 and 500° C. (673 and 773 K). The solid symbols represent the measured values and the dashed lines represent the thermodynamically expected values. The cell provided potentials that coincided to within +/−1 mV with the theoretical values, irrespective of whether a body of Na₂SO₄, Na₂CO₃, Na₂SiO₃, NaF, NaCl or CaF₂ was used in between the two Na-β-alumina bodies.

FIG. 7 is a graph showing the time-dependent cell potential of the cell in FIG. 4 when responding to a variation of the CO₂ and O₂ concentrations of the measured gas, using a Na₂SO₄ body in between the two Na-β-alumina bodies. The gas composition was changed from 0.0034% by volume CO₂ and 21% by volume O₂ to 45% by volume CO₂ and 11% by volume O₂, the balance gas was argon, and the temperature was 550° C. (823 K). The dotted curve represents the measured values and the dashed lines represent the theoretically expected values for the two compositions. The double arrow indicates the difference between the two theoretically expected values. The cell potentials measured before and after the change of composition coincided to within +/−1 mV with the theoretical values. Accordingly, the measured cell potential difference during the response of the cell to the variation of the gas composition also corresponded to the theoretically expected one.

FIG. 8 is a schematic cross-section of a fourth embodiment of the invention in the form of a galvanic cell for the sensing of SO₃ and O₂ gases. The structure of the sensor is similar to that of FIG. 3, but in this case the gas 62 contains SO₃ and O₂, the gas sensitive measurement electrode is Na₂SO₄ 64 which is exposed to the gas, and the electronically-conducting contact at the measurement electrode is platinum or gold 66. The chemical activity of Na in the measurement electrode arises from its equilibration with the SO₃ and O₂. The combination of solid ion-conductors comprises a first Na-β-alumina body 68, a Na₂SO₄ body 70, and a second Na-β-alumina body 72. The electronically-conducting contact at the reference electrode is platinum, gold, tungsten, steel, Inconel, a combination thereof, or some other suitable refractory electronic conductor 74, the reference electrode is pure Na metal 76 which is separated from the environment by means of a glass seal 78. The chemical activity of Na in the reference electrode is unity. The metal leads, or lead wires, coupling the electronically-conducting contacts of the two electrodes to the electrometer 84 are of platinum, gold, tungsten, steel, Inconel, a combination thereof, or some other suitable refractory electronic conductor 80, 82. The Na-β-alumina body 72 that contains the reference electrode is gastight.

FIG. 9 is a graph showing the cell potential of the cell in FIG. 8 as a function of the SO₃ concentration of the measured gas. In experiments, the SO₃ concentration was varied between 5 and 900 ppm by volume, the O₂ concentration was fixed at 14% by volume, the balance gas was argon, and the temperature was 550° C. (823 K). The solid symbols represent the measured values and the dashed lines represent the thermodynamically expected values. The cell provided potentials that coincided to within +/−1 mV with the theoretical values.

In the inventors' experiments several sensors embodying the invention were assembled and tested with respect to their ability to sense molecular gases. The synthesis of the components and their assembly into a sensor are now described.

Na-β-alumina powders were prepared through a solid-state chemical route. The synthesis included drying Na₂CO₃ and γ-AlOOH (Boehmite) powders, mixing appropriate quantities of the powders, wet milling of the powders in distilled water using Al₂O₃ milling spheres, drying, sieving, calcining at 1400° C. in air for 8 h, re-grinding in distilled water and in the presence of an organic binder/plasticiser system such as PVA/PEG (poly-vinyl-alcohol/poly-ethylene-glycol), and again drying and sieving. Discs were prepared by uniaxial pressing. Open-ended tubes were prepared either by isostatic pressing in suitable rubber moulds, or by pressing rectangular bars and drilling cavities into them subsequent to sintering. Sintering of the pressed bodies was performed at temperatures of 1500 to 1600° C. in air for durations of 6 to 30 min. X-ray diffraction analysis proved the successful synthesis of both single-phase material of composition Na₂O.11Al₂O₃ and two-phase material composed of Na₂O.5Al₂O₃ and Na₂O.11Al₂O₃. Both materials were appropriate for use as the ceramic solid metal-ion-conductor.

In each case, a working electrode was prepared by first painting a quantity of a commercial platinum ink or gold ink onto one side of a Na-β-alumina disc. This was heated at 900° C. in ambient air for 2 h. To form a measurement electrode for CO₂ sensing, the platinum or gold coating formed was impregnated with a Na₂CO₃-containing ethanol slurry and dried at 150° C. in air for several hours. To form a measurement electrode for SO₃ sensing, the platinum or gold coating was impregnated with a Na₂SO₄-containing ethanol slurry and dried at 150° C. in air for several hours. To form a measurement electrode for NO₂ sensing, the platinum or gold coating was impregnated with a NaNO₃-containing ethanol slurry and dried at 150° C. in air for several hours. To form a measurement electrode for Cl₂ sensing, the platinum or gold coating was impregnated with a NaCl-containing ethanol slurry and dried at 150° C. in air for several hours.

To form a Na metal reference electrode, a quantity of typically 1 to 3 mg of pure Na metal was placed at the lower end of the inner cavity within an open-ended Na-β-alumina tube. The tube then had a hermetical seal formed at its upper end that consisted of a sealing glass resistant to Na. The sealing glass was an oxide mixture composed of, exclusively, CaO, Al₂O₃, BaO and B₂O₃. The seal was formed by placing either a quantity of glass powder or a suitably-shaped solid glass body on top of the open-ended Na-β-alumina tube, heating this arrangement to slightly above the melting temperature of the glass in an atmosphere of dry argon or other inert gas, and then cooling it down to again. Glasses composed of the above oxides have melting points as low as around 800° C. and are therefore useable in conjunction with elemental Na.

Salt ion-conductors were prepared and applied in contact with the ceramic ion-conductor in different manners to form different sensors for testing: (1) as a body of the pure material, (2) as an infiltrate within a body of a porous support structure of a different chemical composition, or (3) as a surface film on the ceramic ion-conductor.

In the first approach, discs of the salt solid ion-conductor were prepared by uniaxial pressing of powders of salts such as Na₂SO₄, Na₂CO₃, Na₂SiO₃, NaF, NaCl or CaF₂, followed by sintering at temperatures below their melting points for several hours. A typical sintering temperature was 800° C., and a typical sintering time was 8 h.

In the second approach, refractory ceramic discs made of materials such as MgO, Al₂O₃, ZrO₂ or Y₂O₃ and with a significant degree of continuous open porosity, were infiltrated in vacuum with molten salts such as Na₂SO₄, Na₂CO₃, Na₂SiO₃, NaF, NaCl or CaF₂, so that after cooling a continuous path of solidified salt was present throughout the entire refractory ceramic disc.

In the third approach, the salt ion-conductor was formed through a chemical reaction between the surface of the Na-β-alumina disc or blind-ended tube (i.e. the ceramic ion-conductor) and a suitable gas, such as CO₂, SO₂ or Cl₂. The gas contained between a few ppm and 100% of one or more of these components. O₂ may be present in the gas when CO₂ or SO₂ are used, but not when Cl₂ is used. Chemically inert diluent gases such as nitrogen, argon or others may be present or absent in all cases.

To form each sensor, a disc of Na-β-alumina carrying a measurement electrode and constituting the first ceramic solid metal-ion-conductor, a disc comprising Na₂SO₄, Na₂CO₃, Na₂SiO₃, NaF, NaCl or CaF₂ constituting the salt solid ion-conductor, and a blind-ended tube of Na-β-alumina containing a Na metal reference electrode and constituting the second ceramic solid metal-ion-conductor, were brought into contact with each other, in this order, in a custom-built quartz jig. (In the third approach described above, the disc comprising the salt ion-conductor is the layer formed on the surface of the Na-β-alumina disc or blind-ended tube.) As indicated in the figures, the measurement electrode faced away from the series arrangement of the three solid ion-conductors and for exposure, in use, to the gas to be analysed, and the reference electrode faced away from the series arrangement of the three solid ion-conductors in the opposite direction and was protected from the gas by a seal.

Experiments for the sensing of CO₂ and O₂ were carried out with a galvanic cell having the structure shown in FIG. 4 that included Na₂CO₃ as the measurement electrode, Na-β-alumina as the ceramic solid metal-ion-conductor and Na₂SO₄ as the salt solid ion-conductor. Cell potentials were measured between the measurement electrode and the reference electrode with a high-impedance electrometer. Extensive long-term studies were performed with gas mixtures containing up to 1% by volume of CO₂ and up to 1% by volume of O₂, as it is considered that the type of sensor described is expected to have its main application in the field of trace analysis. In one set of experiments, the CO₂ concentration was varied between 0.01 and 1% by volume, the O₂ concentration was constant at 1% by volume, the balance gas was argon, and the temperature was between 400 and 500° C. (673 and 773 K). The cell potentials measured under these conditions were found to be identical to those calculated from published thermodynamic data to within +/−1 mV. FIG. 5 shows the results. In another set of experiments, the O₂ concentration was varied between 0.01 and 1% by volume, the CO₂ concentration was constant at 1% by volume, the balance gas was argon, and the temperature was between 400 and 500° C. (673 and 773 K). The cell potentials measured were again found to be identical to those calculated from published thermodynamic data to within +/−1 mV. FIG. 6 shows the results.

In an alternative arrangement, experiments for the sensing of CO₂ and O₂ were carried out with a galvanic cell having the structure shown in FIG. 4 that included Na₂CO₃ as the salt solid ion-conductor. Other conditions were as described above. Identical cell potentials were measured.

In a further alternative arrangement, experiments for the sensing of CO₂ and O₂ were carried out with a galvanic cell having the structure shown in FIG. 4 that included Na₂SiO₃ as the salt solid ion-conductor. Other conditions were as described above. Identical cell potentials were measured.

In yet a further alternative arrangement, experiments for the sensing of CO₂ and O₂ were carried out with a galvanic cell having the structure shown in FIG. 4 that included CaF₂ as the salt solid ion-conductor. Other conditions were as described above. Identical cell potentials were measured.

The time dependence of the sensor response to variations in the composition of the gas was investigated with two gases of very different CO₂ concentrations, so as to achieve a quantitatively larger change in cell potential. In the experiment, response behaviour was measured by quickly changing the composition of the gas from 0.0034% by volume CO₂ and 21% by volume O₂ to 45% by volume CO₂ and 11% by volume O₂ (balance gas argon) at the temperature of 550° C. (823 K) and following the cell potential as a function of time. The response was fast with 90% of the total cell potential change achieved within less than 5 min. FIG. 7 shows a typical result.

The stability of the cell potential over time was ascertained by measuring the cell potential as a function of time at a constant gas composition and a constant temperature. Cell potentials remained constant under these conditions to within +/−1 mV over extended periods of time, with six weeks being the longest single measurement performed. There was no indication that longer measuring times would lead to cell-potential drift.

Experiments found that the preferred temperature range for gas sensing with the above type of galvanic cell is between 300 and 600° C. Temperatures lower than 300° C. are less preferred, because these led to deviations between the measured and the thermodynamically expected cell potential, probably because of an increase in cell impedance. Temperatures higher than 600° C. are less preferred, because of their adverse impact on the sensor durability.

Experiments for the sensing of SO₃ and O₂ were carried out with a galvanic cell having the structure shown in FIG. 8 that included Na₂SO₄ as the measurement electrode, Na-β-alumina as the ceramic solid metal-ion-conductor and Na₂SO₄ as the salt solid ion-conductor. Cell potentials were measured between the measurement electrode and the reference electrode with a high-impedance electrometer. In the experiment, the SO₃ concentration was varied between 5 and 900 ppm by volume, the O₂ concentration was constant at 14% by volume, the balance gas was argon, and the temperature was 550° C. (823 K). The cell potentials measured under these conditions were found to be identical to those calculated from published thermodynamic data to within +/−4 mV. FIG. 9 shows the results.

Control experiments were performed with galvanic cells of the above type but without the salt solid ion-conductor in between the two ceramic solid metal-ion-conductors, and these yielded measured cell potentials that were, without exception, lower by several hundreds of millivolts than the thermodynamically-expected values. A clear response behaviour to changes in the concentration of the target species in the gas was detected, but the response was never fully quantitative and was typically followed by significant drift.

It is understood that the measurements described above have been obtained with specific embodiments of the present invention. Various other geometric arrangements and materials combinations may be realised without departing from the scope and spirit of the present invention. A few of these embodiments are now named. All sensor components may be used in arbitrary geometries other than those mentioned. A combination of more than three solid ion-conductors may be used. Ceramic solid metal-ion-conductors other than Na-β-alumina may be used, most importantly NASICON, but also many others including those mentioned afore. Salt solid ion-conductors other than Na₂SO₄, Na₂CO₃, Na₂SiO₃, NaF, NaCl or CaF₂ may be used. The salt solid ion-conductors may not only be used in their pure forms but also in mixtures or in their partially-substituted forms; for example, Na₂SO₄ may contain La and/or Y as a partial substitute for Na, and Na₂SO₄ may contain WO₄²⁻ ions as a partial substitute for SO₄²⁻. Both the ceramic solid ion-conductor and the salt solid ion-conductor may contain admixtures of non-conducting second phases, such as ZrO₂, Y₂O₃, SiO₂ or others. Reference electrodes other than pure Na metal may be used, for example, alloys, intermetallics or compounds containing Na, either protected from the gas by a seal or open to the gas.

1. A sensor for a molecular gas, comprising;
a measurement electrode;
a reference electrode; and
an electrolyte in electrical contact with the measurement electrode and the reference electrode;
in which the electrolyte comprises, electrically connected in series between the measurement electrode and the reference electrode;
a first ion-conductor, which is a ceramic, or glass, solid ion-conductor; and
a second ion-conductor, which comprises an ion-conducting salt.

2. A sensor according to claim 1, in which the second ion-conductor is a solid ion-conductor.

3. A sensor according to claim 1 or 2, in which the measurement electrode comprises an auxiliary phase exposable, in use, to a measurand gas comprising the molecular gas, and an electronic contact in electrical contact with the auxiliary phase.

4. A sensor according to claim 1, 2 or 3, in which the first ion-conductor has a first ion as its mobile species and the second ion-conductor has a second ion as its mobile species, in which the first and second ions are the same ions.

5. A sensor according to claim 1, 2 or 3, in which the first ion-conductor has a first ion as its mobile species and the second ion-conductor has a second ion as its mobile species, in which the first and second ions are different ions.

6. A sensor according to any preceding claim, in which the first ion-conductor is a metal-ion-conductor, and preferably an alkali-metal ion, alkaline-earth-metal ion, copper ion, silver ion or rare-earth-metal ion conductor.

7. A sensor according to any preceding claim, in which the first ion-conductor is a sodium ion-conductor.

8. A sensor according to any preceding claim, in which the first ion-conductor comprises Na-β-alumina or NASICON, or comprises one or more materials selected from the group consisting of Li-β-alumina, Na-β-alumina, K-β-alumina, Mg-β-alumina, Ca-β-alumina, Sr-β-alumina, Ba-β-alumina, Cu-β-alumina, Ag-β-alumina, rare earth metal β-aluminas, LISICON, CuSiCON and oxide glass.

9. A sensor according to any preceding claim, in which the second ion-conductor comprises a salt containing one or more cations selected from the group consisting of lithium, sodium, potassium, rubidium, caesium, magnesium, calcium, strontium, barium, lanthanum, yttrium and other rare-earth metals.

10. A sensor according to any preceding claim, in which the second ion-conductor comprises a salt containing one or more anions selected from the group consisting of carbonate, nitrate, sulphate, meta-silicate, ortho-silicate, ortho-phosphate, fluoride, chloride and bromide.

11. A sensor according to any preceding claim, in which the second ion-conductor comprises one or more materials selected from the group comprising Na₂CO₃, Na₂SO₄, Na₂SiO₃, NaF, NaCl, CaF₂, a mixture or combination of these materials, and any of these materials in partially-substituted form.

12. A sensor according to any preceding claim, in which the second ion-conductor comprises a solid body.

13. A sensor according to any preceding claim, in which the second ion-conductor comprises an infiltrate within a porous support structure, the porous support structure preferably comprising a refractory ceramic material, such as MgO, Al₂O₃, ZrO₂ or Y₂O₃.

14. A sensor according to any preceding claim, in which the second ion-conductor comprises a surface modification of the first ion-conductor, preferably formed by exposing the surface to a gas.

15. A sensor according to claim 14, in which the second ion-conductor comprises one or more materials selected from the group consisting of Na₂CO₃, Na₂SO₄ and NaCl, preferably prepared by exposing the first ion-conductor to a gas containing one or more materials selected from the group consisting of CO₂, SO₂, Cl₂, O₂ and inert gases.

16. A sensor according to any preceding claim, in which the electrolyte comprises a third ion-conductor, which is a ceramic, or glass, solid ion-conductor.

17. A sensor according to claim 16, in which the second ion-conductor is in electrical contact with the third ion-conductor and separates the first and third ion-conductors.

18. A sensor according to claim 16 or 17, in which the first ion-conductor has the first ion as its mobile species, the second ion-conductor has the second ion as its mobile species, and the third ion-conductor has a third ion as its mobile species, in which the first, second and third ions are all different ions, or two of the first, second and third ions are the same ions, or all of the first, second and third ions are the same ions.

19. A sensor according to any of claims 16 to 18, in which the third ion-conductor is a metal-ion-conductor, and preferably an alkali-metal ion, alkaline-earth-metal ion, copper ion, silver ion or rare-earth-metal ion conductor.

20. A sensor according to any of claims 16 to 19, in which the third ion-conductor is a sodium ion-conductor.

21. A sensor according to any of claims 16 to 20, in which the third ion-conductor comprises Na-β-alumina or NASICON, or comprises one or more materials selected from the group consisting of Li-β-alumina, Na-β-alumina, K-β-alumina, Mg-β-alumina, Ca-β-alumina, Sr-β-alumina, Ba-β-alumina, Cu-β-alumina, Ag-β-alumina, rare earth metal β-aluminas, LISICON, CuSiCON and oxide glass.

22. A sensor according to any of claims 16 to 21, in which the first and third ion-conductors comprise the same materials.

23. A sensor according to any of claims 16 to 22, in which the first and third ion-conductors comprise different materials.

24. A sensor according to any of claims 16 to 23, in which the second ion-conductor comprises a surface modification of the third ion-conductor, preferably formed by exposing the surface to a gas.

25. A sensor according to claim 24, in which the second ion-conductor comprises one or more materials selected from the group consisting of Na₂CO₃, Na₂SO₄ and NaCl, preferably prepared by exposing the third ion-conductor to a gas containing one or more materials selected from the group consisting of CO₂, SO₂, Cl₂, O₂ and inert gases.

26. A sensor according to any of claims 3 to 25, in which the auxiliary phase comprises a binary or ternary compound, or a mixture of such compounds, that can chemically equilibrate with the molecular gas or gases in the measurand gas, and that comprises the metal whose ion is mobile in the first ion-conductor.

27. A sensor according to claim 26, in which the binary or ternary compound, or mixture of such compounds, can chemically equilibrate with one or more of CO₂, SO₃, NO₂, and Cl₂.

28. A sensor according to claim 26 or 27, in which the auxiliary phase comprises Na₂CO₃ (sodium carbonate) for the sensing of CO₂ and O₂, Na₂SO₄ (sodium sulphate) for the sensing of SO₃ and O₂, NaNO₃ (sodium nitrate) for the sensing of NO₂ and O₂, or NaCl (sodium chloride) for the sensing of Cl₂.

29. A sensor according to any preceding claim, in which the reference electrode comprises a unary, binary or ternary compound, or mixture of such compounds, that provides a predetermined chemical activity of the metal whose ion is mobile in the second ion-conductor.

30. A sensor according to claim 29, in which the metal comprises Na, preferably elemental Na.

31. A sensor according to claim 29 or 30, in which the reference electrode is protected from surrounding gas by a seal.

32. A sensor according to claim 29 or 30, in which the reference electrode is open to surrounding gas.

33. A sensor according to any preceding claim, in which the electrolyte comprises more than two ceramic, or glass, solid ion-conductors in electrical contact in series between the auxiliary phase and the reference electrode.

34. A sensor according to any preceding claim, in which the electrolyte comprises more than one ion-conductor comprising a salt, in electrical contact in series between the measurement electrode and the reference electrode, the or each ion-conductor being spaced from each of the measurement electrode and the reference electrode by one of the ceramic, or glass, solid ion-conductors.

35. A sensor according to any preceding claim, further comprising a heater to raise the temperature of the sensor to a predetermined operating temperature.

36. A method for sensing a molecular gas, comprising the steps of;
exposing a measurement electrode to a measurand gas comprising the molecular gas;
generating a reference potential at a reference electrode;
allowing ionic conduction through an electrolyte between the measurement electrode and the reference electrode, the electrolyte comprising a first ion-conductor, which is a ceramic, or glass, solid ion-conductor, and a second ion-conductor, which comprises an ion-conducting salt, electrically connected in series between the measurement electrode and the reference electrode; and
measuring a potential difference between the measurement electrode and the reference electrode.

37. A method according to claim 35, in which the second ion-conductor comprises a surface modification of the first ion-conductor, preferably formed by exposing the surface to a gas.

38. A method according to claim 36 or 37, in which the electrolyte comprises a third ion-conductor, which is a ceramic, or glass, solid ion-conductor.

39. A method according to claim 38, in which the second ion-conductor is in electrical contact with the third ion-conductor and separates the first and third ion-conductors.

40. A method according to claim 38 or 39, in which the second ion-conductor comprises a surface modification of the third ion-conductor, preferably formed by exposing the surface to a gas.

41. A method according to any of claims 36 to 40, in which the measurement electrode comprises an auxiliary phase comprising a binary or ternary compound, or a mixture of such compounds, that chemically equilibrates with the molecular gas or gases in the measurand gas, and that comprises the metal whose ion is mobile in the first ion-conductor.

42. A method according to claim 41, in which the binary or ternary compound, or mixture of such compounds, chemically equilibrates with one or more of CO₂, SO₃, NO₂, and Cl₂.

43. A method according to any of claims 36 to 42, in which the reference electrode comprises a unary, binary or ternary compound, or mixture of such compounds, that provides a predetermined chemical activity of the metal whose ion is mobile in the second ion-conductor.

44. A method according to any of claims 36 to 43, comprising the step of protecting the reference electrode from surrounding gas by a seal.

45. A method according to any of claims 36 to 43, comprising the step of exposing the the reference electrode to surrounding gas.

46. A method for making a sensor for sensing a molecular gas, comprising the steps of;
arranging an electrolyte comprising a first ion-conductor, which is a ceramic, or glass, solid ion-conductor, and a second ion-conductor, which comprises an ion-conducting salt, electrically connected in series between a measurement electrode and a reference electrode.

47. A method according to claim 46, comprising the step of forming the second conductor as a surface modification of the first ion-conductor, preferably formed by exposing a surface of the first ion-conductor to a gas.

48. A method according to claim 46 or 47, comprising the step of additionally arranging a third ion-conductor, which is a ceramic, or glass, solid ion-conductor, electrically connected in series between the measurement electrode and the reference electrode.

49. A method according to claim 48, comprising the step of forming the second ion-conductor as a surface modification of the third ion-conductor, preferably formed by exposing a surface of the third ion-conductor to a gas.

50. A sensor for sensing a molecular gas substantially as described herein, with reference to the accompanying drawings.

51. A method for sensing a molecular gas substantially as described herein, with reference to the accompanying drawings.

52. A method for making a sensor for sensing a molecular gas substantially as described herein, with reference to the accompanying drawings.
